(19) United States
(12) Patent Application Publication
(10) Pub. No.: US 2009/0254075 A1
(43) Pub. Date: Oct. 8, 2009

(54) Surgical Instrument

(75) Inventors: Adrian Paz, Petach Tikva (IL); Roni Shabat, Kibutz Izrael (IL)

Correspondence Address: Avia Kafri, 22Shderot Chen St., Tel Aviv, 64166, IL

(73) Assignee: Ultrasurge Technologies Ltd., Migdal Haemek (IL)

(21) Appl. No.: 11/989,104

(22) PCT Filed: Jul. 26, 2006
(86) PCT No.: PCT/IL06/00869

§ 371 Date: Jan. 22, 2008

Related U.S. Application Data
(60) Provisional application No. 60/702,304, filed on Jul. 26, 2005.

(51) Int. Cl. A61B 18/04

(52) U.S. Cl. 606/28

(57) ABSTRACT

A surgical instrument suitable for endoscopic operations having a gripping handle, tubular body and an operational tip. Jetting outlets laterally disposed at the operational tip provides for jetting pressurized liquid delivered by the tubular body. The jetting outlets are configured as to emit converging jets. Two or more jets converge at a convergence point laterally displaced at a predefined distance from the surface of the operational tip. The operational tip includes a heating member having an active face for contact heating a bleeding tissue. A rotating mechanism provides for independently rotating the jetting outlets and the active surface of the heating member relative to the gripping handle. A method for regulating the operating temperature of the heating member is provided.

### FIELD OF THE INVENTION

The present invention relates in general to surgical instruments and in particular the present invention to surgical instruments utilizing a liquid jet for cutting and removing tissues and thermally sealing blood vessels.

### BACKGROUND OF THE INVENTION

Heating of body tissue is done to achieve a variety of effects such as cutting, coagulation, and ablative necrosis. The effect obtained depends upon the temperature to which the tissue is heated. At temperatures above 70° C. (degrees Celsius), coagulation occurs, leading to hemostasis due to shrinkage of blood vessels. At temperatures above 100° C., tissue water evaporates leading to desiccation of the tissue. A tubular body organ can be sealed by either “coagulation” or vessel “welding”.

The term coagulation refers to a process in which tissue cells are ruptured and desiccated. Tissue welding involves heating tissue to a temperature high enough to liquefy collagen in the tissue (between 70 and 90 degrees Celsius) so that the collagen cross-links and reforms in a fused mass. Coagulation is usually sufficient to seal small vessels; larger vessels usually require welding.

Electrosurgery has been used to heat tissue. In accordance with this method, conducting an electric current through the tissue generates heat. Electrosurgical devices are commonly used to achieve coagulation and tissue welding. U.S. Pat. Nos. 6,858,028 and 6,398,779 disclose electrosurgical devices for sealing a blood vessel. Electrosurgery is particularly suitable in narrow spaces such as encountered in endoscopic operations. The electrical current permits cutting and removing tissues and coagulation of blood vessels. However, this method has significant drawbacks. Conduction of an electrical current through tissue causes an electrical burn leading to thermal damage to the normal tissues extending beyond the operative site. Heating tissue to high temperatures leads to tissue charring and desiccation that prevents optimal sealing of larger blood vessels and may lead to intraoperative or postoperative bleeding.

Internal organs are sensitive, especially to extreme temperatures and distension and less so to cutting or piercing. Therefore, during cutting and coagulating tissues the extreme heat generated by the electrosurgical units is very painful necessitating a deeper level of anesthesia such as regional or general anesthesia. Electrical burn can cause significant postoperative suffering and complaints leading to a long convalescence. Cutting and removing tissues by generating high temperature can lead to tissue distortion and difficult histological evaluation.

Methods for cutting tissues employing liquid jets are known. World patent application WO 9,639,954A1 discloses a surgical instrument for delivering a pressurized stream of liquid as a coherent jet. The disclosed instrument includes a jet orifice that can be oriented axially, transversally or obliquely. Deflector slidably, or pivotally, attached to the jet orifice provides for controlling the length of the liquid jet and therefore the size of the cut tissue. Both the jet orifice and the deflector are remotely steerable enabling a user to selectively alter the direction of cutting without having to remove the instrument from the surgical site.

U.S. Pat. No. 6,960,182 discloses a variety of surgical instruments forming a liquid jet, which are useful for surgical procedures. The disclosed instruments include a pressure lumen and an evacuation lumen. The pressure lumen includes at least one nozzle for forming a liquid jet, whereas the evacuation lumen includes a jet-receiving aperture. The nozzles and jet-receiving aperture are positioned relative to each other such that the liquid comprising the liquid jet and any tissue or material entrained by the liquid jet is evacuated through the jet-receiving aperture. Some of the disclosed instruments also utilize a liquid jet-driven rotor mechanism for driving rotation of a rotatable shaft. Surgical components drivable by the rotatable shaft can be used for performing various surgical tasks such as grinding, abrading, cutting, drilling, polishing, or screwing.

Such disclosed instruments necessitate mechanical means, such as a deflector, or an evacuating tube for blocking the liquid jet and thereby limiting its range. Furthermore these instruments do not provide integral means for hemostasis.

There is a need for a surgical device that can perform blood vessel sealing (hemostasis) at temperature bellow the tissue charring and desiccation temperatures.

There is a need for a surgical device that can perform hemostasis without causing thermal tissue injury beyond the site of the blood vessel.

There is a need for a surgical device that can perform hemostasis safely.

There is a need for a surgical device that can cut and remove tissue precisely, without causing thermal and/or mechanical injury to remaining tissue.

There is a need for a surgical device that has a depth control during cutting and removing tissues.

There is a need for a surgical device that can preferentially remove softer tissue and spare harder tissues.

There is a need for a surgical device that can remove tissue for histological examination without causing thermal damage and tissue alteration.

There is a need for a surgical device that can perform both tissue cutting and hemostasis concomitantly or successively and at will and that is easy to manipulate.

There is a need for surgical device that has a slender shaft that can be easily manipulated in narrow spaces such as during endoscopic procedures.

There is a need for a surgical device that can be used under local anesthesia with sedation.

There is a need for a surgical device that can be used in the office for endoscopic procedures such as endoscopic prostatectomy.

### BRIEF DESCRIPTION OF THE DRAWINGS

FIG. 1A is a side looking view of a surgical instrument of the present invention;

FIG. 1B is a partially fragmented side looking view of the operational tip shown in FIG. 1;

FIG. 2A is a longitudinal sectional view of the tubular body of a surgical instrument according to a preferred embodiment of the present invention;

FIG. 2B is a partially fragmented elevational view of a surgical tip according to a preferred embodiment of the present invention;

FIG. 3A is an elevational view of a surgical tip according to another preferred embodiment of the present invention;

FIG. 3B is a partially fragmented elevational view of the surgical tip shown in FIG. 3A;

FIG. 4A is a schematic description showing the arrangement of jetting outlets of an operational tip according to a preferred embodiment of the present invention;

FIGS. 4B, 4C and 4D are schemes respectively showing arrangements of the jetting outlets of an operational tip according to different embodiment of the present invention;

FIG. 5A is a sectional view along the line AA of the operational tip shown in FIG. 2B;

FIG. 5B is a sectional view along the line BB of the operational tip shown in FIG. 2B;

FIG. 5C is a sectional view along the line CC of the operational tip shown in FIG. 2B;

FIG. 5D is a schematic description of the operational tip shown in FIG. 2B.

### DETAILED DESCRIPTION OF THE INVENTION

In accordance with the present invention a surgical instrument employing liquid jets and/or contact heating is provided. The surgical instrument of the invention provides for cutting and removing soft tissues and for carving and shaping bone such as for fitting in prosthesis. The instrument includes an operational tip disposed at the distal end of a slender tubular body attached to a gripping handle. It is especially accommodated for endoscopic procedures such as removing hyperplastic prostate tissue. Cutting and/or mincing of targeted tissue is effected by a plurality of liquid jets ejected laterally from jetting outlets located at a side wall of the operational tip. A heating member disposed at the distal end of the operational tip provides for stopping bleeding by contact heating.

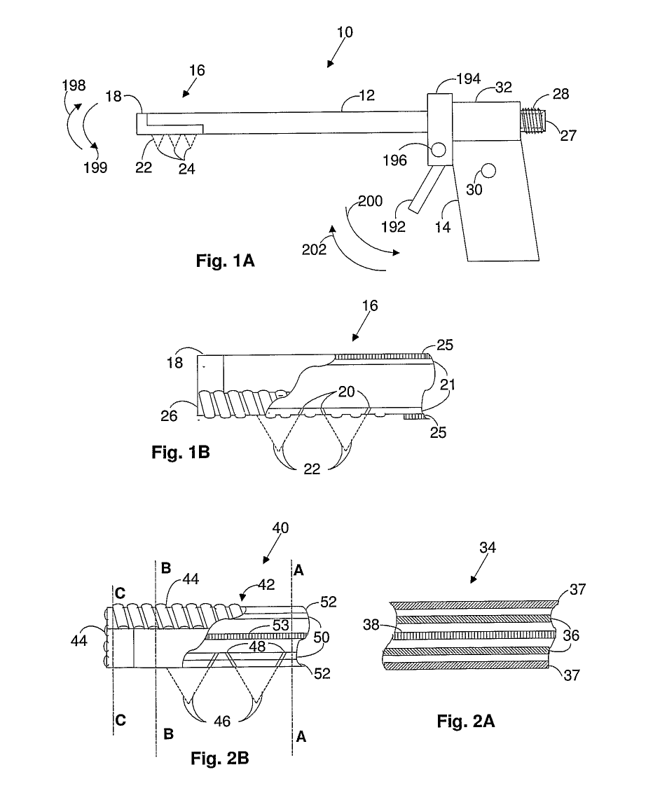

Reference is now made to FIGS. 1A-1B showing respectively a side view of a surgical instrument of the present invention and a fragmented side view of the operational tip. Surgical instrument 10 includes slender tubular body 12 and gripping handle 14. Hollow operational tip 16 is disposed at the distal end of slender tubular body 12, such that its lumen forms a continuum with the lumen of tubular body 12. Operational tip 16 includes heating member 18 and a plurality of jetting outlets 20 disposed at a wall of inner tube 21. A pressurized liquid conducted by the tubular body is ejected at high speeds from the orifices of these jetting outlets. These jetting outlets and their orifices are configured such that pairs of adjacent jets 22 converge at converging points 24 displaced laterally from the operational tip.

Inner tube 21 is surrounded by thermally insulating layer 25 except for its distal end that is only partially covered. The uncovered segment of inner tube 21 constitutes active face 26 of heating member 18. An active face according to the invention has a good thermal connection with heating member 18. Optional ridges located on the surface of active face 26 provide for enlarging its area as is further described infra. The inner tube, which is typically made of stainless steel and is substantially thin, provides for delivering the pressurized liquid. Insulting layer 25 is typically made of Teflon® or any thermal insulating material that is biocompatible. The insulating layer may include embedded cavities containing thermal insulating materials, or phase changing materials (PCMs), such as semi crystalline fats or hydrocarbons, gas, or vacuum. Encapsulated porous materials can be used as well. Alternatively cavities are disposed between a sheath of insulating layer and inner tube 21.

Liquid inlet 27 is provided with threading 28 in order to provide for a connection to an external source of liquid. Operating switch 30 provides for activation of an electrical heating element, not shown, embedded in heating unit 32. The heating is regulated by means of a regulating device, not shown, as is known in the art for retaining a constant predefined temperature of the conducted liquid within a range of temperatures above 45 degrees Celsius and preferably less then the boiling temperature of the liquid utilized. (In cases of aqueous solution this temperature is preferably not higher than 100 degrees Celsius.) The total capacity of the slender tubular body and the operational tip of the invention is typically lower than 0.5 cm³ and a typical flow rate of the liquid is 2 cm³/sec. Therefore a moderate heating power is sufficient for raising the temperature of the heating member to its operating value within a few seconds.

Optionally, heating of the conducted liquid is induced by electromagnetic radiation in the range of radiofrequencies, or microwaves, or by transmission of ultrasonic vibrations, or by employing thermoelectric cooling or heating. Alternatively, a heated liquid may be provided from an external source. Gripping handle 14 and any hoses connected to it are thermally insulated to prevent heat injuries to the operator or the patient.

Reference is now made to FIG. 2A showing a longitudinal sectional view of a segment of slender tubular body 34 according to a preferred embodiment of the present invention. Slender Tubular body 34 has an inner tube whose wall 36 is typically made of stainless steel. The volume of the space separating between wall 36 and external wall 37 of slender tubular body 34 is preferably vacuumed, filled with an isolating material, or used for delivering liquid. The temperature of the surface of the tubular body has to be retained below a predetermined temperature such that a risk of thermal injury caused to tissues with which it is engaged is minimized. In some embodiments of the invention the slender tubular body is actively cooled by circulating or flushing cooling fluids through cavities embedded in the insulating material, or through the space disposed between external wall 37 and wall 36. Optionally, the slender tubular body is cooled by means of thermoelectric cooling (TEC).

Shaft 38 provides for rotating the heating member as is further described infra.

Operational Tip

Reference is made to FIG. 2B in which a partially fragmented side view of an operational tip according to a preferred embodiments of the present invention is shown. Operational tip 40 consists of the distal segment of tubular body 42 onto which heating member 44 is attached. Axes 46 are the axes of the liquid jets and are referred hereinafter by jet axes. The orifices and channels leading to the orifices of jetting outlets 48 are typically axially symmetric, in that their slanting angle is equal and the jet axes converge at a converging point, laterally displaced from operational tip 40. Such pair of jetting outlets whose jet axes converge at a point, are referred to hereinafter by converging jetting outlets. Inner tube 50 is surrounded with thermally insulating layer 52. Shaft 53 provides for rotating heating member 44 relative to tubular body 42.

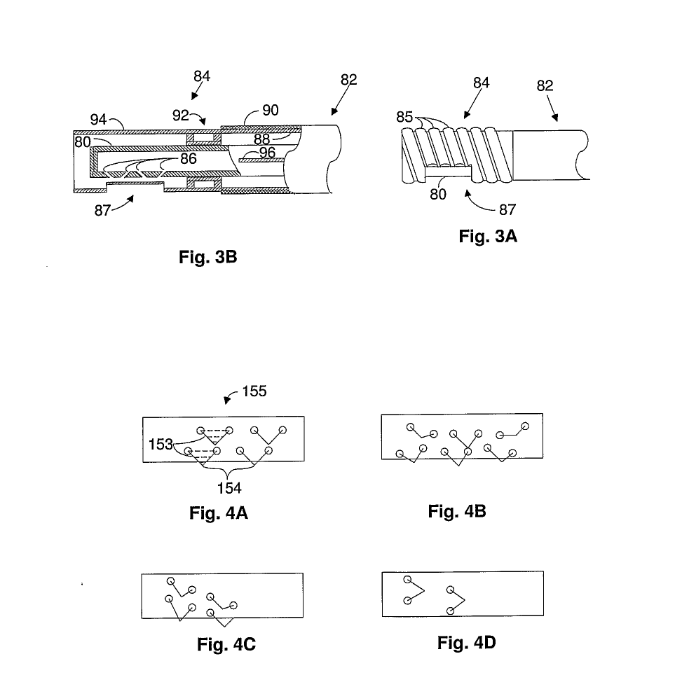

Reference is now made to FIGS. 3A-3B in which an elevational view and a partially fragmented side looking view of an operational tip according to another preferred embodiment of the present invention are respectively shown. The distal end of inner tube 80 extends from tubular body 82 and constitutes the liquid delivering member of operational tip 84. Ridges 85 disposed on the surface of operational tip 84 provides for enlarging its area. Jetting outlets 86 are laterally disposed on inner tube 80 facing window 87, which enables emitted jets to pass through and hit the targeted tissue. External tube 88 coaxially surrounds inner tube 80 along the entire length of tubular body 82. Optionally insulating layer 90 surrounds external tube 88. Heating member 92 from which cylindrical active face 94 distally extends is firmly connected to external tube 88 and slidingly attached to inner tube 80, such that it seals the distal end of the volume spacing between them. (The ridges disposed on external surfaces of active face 94 and heating member 92 are not shown in FIG. 3B.) External tube 88, heating member 92 and active face 94 together form an integral rigid body rotatable around inner tube 80. Optionally wall 96 divides this volume into two segments connected close to heating member 92. The walls of heating member 92 and active face 94 are made of a good thermal conducting material optionally coated with biocompatible material as is further described below. The temperature of heating member 92 and active face 94 is typically regulated by the emitted liquid jets when external tube 88 is such rotated that active face 94 blocks them. Optionally such temperature is also regulated by means of heated or cooled liquid delivered through the two segments of volume separating between inner tube 80 and external tube 88. In such a case two additional pipes, not shown, one for delivering liquid and another for evacuating them, to, or from the heating member are provided. Heating such temperature regulating liquid is induced by the heating unit described hereinabove.

Optionally heating member 92 contains a heat buffer and or includes embedded electrical heating element, providing for, or assisting in regulating the operating temperature of heating member 92 and active face 94. Transformation from cutting to contact heating is achieved by rotating external tube from an angle in which window 85 is disposed in front of the jetting outlets at a stage of cutting, to an angle in which a wall of the active face blocks the liquid jets prior to, or at, a stage of contact heating.

Reference is now made to FIGS. 4A-4D in which different configurations of jetting outlets according to the present invention are shown. A pressurized liquid ejected from the orifices of a pair of adjacent converging jetting outlets at high speed, constitute virtual planar blade 153. Any two converging jets substantially cancel each other upon collision at converging point 154 transforming into a harmless spray that cannot injure tissue. Converging jetting outlets are configured to form an angle of 10 to 90 degrees and preferably of 45 to 60 degrees from the tubular body. A converging point is normally located at a range of 1 mm to 10 mm laterally to the wall of operational tip 155 and preferably at a range of 2 to 4 mm. Optionally, more than two jets may collide at the same converging point, representing a three dimensional virtual blade. The converging points of various convergent jets sets are located at different lateral distances, or at the same distance, from the surface of the operational tip. The planes in which jet axes of any pair of converging jets are contained are parallel or intersecting as is depicted in FIGS. 4A-4D.

In FIG. 4A a scheme of jetting outlets according to a preferred embodiment of the invention is shown. Operational tip 155 includes four pairs of converging jetting outlets. Jetting axes of two pairs of jetting outlets axially disposed and their respective converging points are contained in one plane longitudinally disposed along the axis of operational tip 155. The planes containing each set of pairs are mutually parallel to the main axis of operational tip 155. Alternatively, planes containing jet axes of colliding jets may intersect each other forming an angle of 10 to 60 degrees and more preferably of 15 to 30 degrees between them. Adjacent pairs of jetting outlets need not be laterally located at the same distance from the distal end of the inner tube such that mechanical integrity of the wall of an operational tip is retained.

In FIG. 4D a scheme of jetting outlets according to another preferred embodiment of the present invention is shown. The orifices of each of the two pairs of jetting outlets are radially disposed, preferably at different distance from the distal surface of the inner tube and radially interleaved, such that the mechanical integrity of the lateral wall of the operational tip is retained up to its maximal extent.

Various other arrangements of the jetting outlets, in which planes containing jet axes intersect at any angle between them or between the planes and the main axis of the operational tip, are in accordance with the present invention. In any of such an arrangement the virtual blades formed by the liquid jets constitute a virtual brush that can be used to remove a layer of a tissue by linearly sweeping it laterally to, or along, the direction of the main axis of the operational tip.

The Heating Member

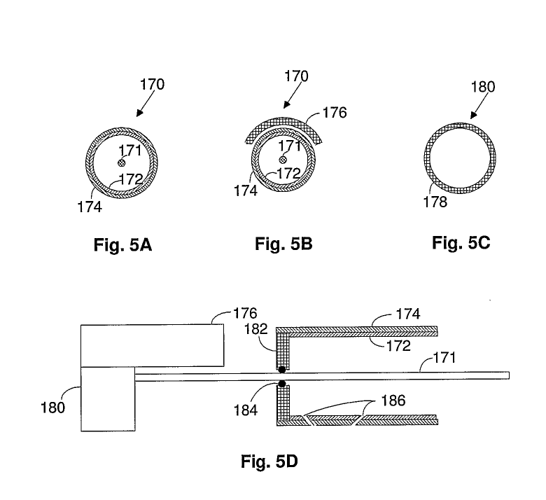

Reference is now made to FIGS. 5A-5D in which sectional views of the operational tip along lines AA, BB and CC shown in FIG. 2B, and a schematic assembly drawing of this operational tip, according to a preferred embodiment of the present invention, are respectively shown. Axial shaft 171 disposed along the axis of inner tube 172 provides for rotating active face 176 relative to insulating layer 174. Wall 178 of the hollow body of heating member 180 and active face 176 are typically made of a good heat conducting metal such as a thin sheath of stainless steel, and/or externally plated with a biocompatible material such as stainless steel or titanium. Such heat conducting surface(s) provide for sealing off of blood vessels when active face 176 or the distal surface of the heating member are pressed against a bleeding tissue. Heating member 180 and active face 176 are rotated relative to the axis of the operational tip into a blocking stage in which the liquid jets are blocked prior to the contact heating of the bleeding tissue. When cutting is desired hollow body 180 and attached active face 176 tare rotated back to the open stage as is shown in FIG. 5D. The walls of heating member 180 and its active face 176 are good heat conductors. Therefore both are primarily heated by the liquid jets impinging on the inner surface of active face 176 when is rotated into a blocking stage. Heat is also conducted by contact to wall 178 and through axial shaft 171. Pressure sustaining bearing 184 provides for rotating heating member by means of axial shaft 171. Jetting outlets 186 are such disposed that their channels pass through wall 172 and isolating layer 174.

Alternatively, the active face of the heating member is firmly attached to the external surface of the inner tube of the operational tip, such that it surrounds the jetting outlets. In such a case transforming from cutting to contact heating requires reducing the level of pressure of the delivered liquid prior to contact heating.

Regulating the Temperature of the Heating Member

The operating temperature of a heating member is typically selected from a range of temperatures such that collagen melting and blood vessel sealing with minimal tissue desiccation and without charring is provided. In order to increase the transfer rate of heat to a tissue, ridges, bulges, peaks, valleys, recesses or depressions are disposed on the external surfaces of the heating member and its active face thereby increasing their area. The heating member optionally contains a thermal or heat buffer such as a phase change material (PCM). PCMs are materials that upon transformation from one phase to another such as from solid crystalline to amorphous solid (solid-solid); solid to liquid, or liquid to gas, absorb or release a substantially large amount of energy concomitantly associated with small changes in temperature. At the transition phases such PCMs act as thermal buffers retaining a relatively constant temperature. Exemplary materials are hydrocarbons with carbon chains preferentially longer than 16 or saturated fatty acids. A PCM may be contained within one or more cavities embedded in the heating member body.

The operations of cutting and mincing of a tissue by means of liquid jets according to the invention are carried out utilizing a cool or warm liquid. However the pressurized liquid is heated prior to contact heating to temperatures hazardous to neighboring tissues. Due to the relatively small dimensions of the heating member and the volume of the inner tube, the heat capacity of the operational tip of the surgical instrument of the invention is considerably low. Therefore a leading time for raising a temperature of the heating member to its operating value and a tailing time for cooling the pressurized liquid and the external surfaces of the operational tip to an operating temperature suitable for cutting operation are in the range of seconds. Therefore a surgeon can alternately and continuously carry out cutting and contact heating. Furthermore, a warm liquid surrounding the distal end of the surgical instrument provides for warming of engaged tissue. In turn the efficiency of contact heating is enhanced due to a relatively low temperature difference between the operating temperature of the heating member and the temperature of the tissue engaged. Therefore the duration of the time interval in which heated liquid is utilized for contact heating decreases and associated thermal hazards are decreased.

Optionally raising a temperature of a heating member of the invention to its operating value is carried out, or assisted, electrically as is known. An electrical heating element and/or TEC embedded in the heating member, or attached to it, which are remotely activated, provides for such heating. One or more temperature sensors embedded in the heating member, a servomechanism, positive temperature conductor (PTC) employed for the heating element, and/or contained PCMs provide for regulating the temperature of the heating member within the desired range.

Operating a Surgical Instrument of the Invention

Liquids typically utilized according to the invention are any sterile physiological liquid such as saline 0.9% solution, Ringer's solution, or other similar biocompatible fluids. Liquids are normally stored in a remote reservoir and are pumped and pressurized by means of an external pump connected with suitable piping to the liquid inlet, or inlets, of the surgical instrument of the invention. A pressure control mechanism as known in the art is provided to activate and regulate the pressure of the liquid during cutting and contact heating phases. Optionally a piston pump is provided and housed within the gripping handle of the surgical instrument.

Reference is again made to FIG. 1A. Lever 192 pivotally connected to gear box 194 provides for rotating tubular body 12. Selector 196 is provided in cases in which the heating member, or its active face, is rotatable independently from, or simultaneously with, the jetting outlets of operational tip 16. Selector 196 is movable between two positions. In the first position, rotating the jetting outlets and the heating member in two opposing directions 198, or 199, is effected by rotating lever 192 in directions 200, or 202 respectively. At a second position only heating member 18 and/or its active face are rotated. Such means for independently rotating the active face of a heating member or the jetting outlets provides for firmly holding griping handle 14 at a substantially fixed orientation while conveniently directing the pressurized jets to the targeted area and/or conveniently transforming from cutting to contact heating. Similarly the inner tube or the external tube together with the heating member and its active face according to the other preferred embodiment described hereinabove are independently rotated by such rotating means. Furthermore rotational sweeping of the virtual brush by itself or in combination with a translational movement along the axis of the operational tip enables the convenient removal of a layer of tissue.

Optionally two independent rotational wheels disposed adjacent to the gripping handle and a selector having two stages substitute the rotational means described hereinabove. When the selector is moved into a first position the wheels are independently rotatable, whereas in the second position the two wheels simultaneously rotate in the same direction. One wheel is connected to the tubular body and the other is connected to the axial shaft disposed within it, or alternatively one wheel is connected to the inner tube and the other to the external tube of the slender tubular body. A slight movement of a finger can induce rotation of any of these wheels, while the gripping handle is retained at the same orientation.

Further Possible Applications

Optional applications include endoscopic removal of bladder tumors, by a similar technique. Similarly the surgical instrument and the method of the invention may be used also for endoscopic removal of uterine mucosa or uterine polyps and during arthroscopy for removal of soft tissue.

A surgical instrument of the invention can be used for endoscopic contact heating. In such a case the jetting outlets are optionally shut off. Obviously a surgical instrument devoid of the heating member is suited for endoscopic cutting and mincing a tissue.

### Example

Endoscopic resection of the prostate employing the surgical instrument according to the preferred embodiment described hereinabove is described in the following. A conventional cystoscope having a central working channel is employed. The cystoscope is introduced through the urethra to the prostatic urethra. The slender tubular body of the surgical instrument is introduced through the working channel of the cystoscope. A physiological sterile liquid such as 0.9% saline is utilized. The liquid is stored at room temperature in a remote container. The liquid is pressurized and jetted through the jetting outlets of the operational tip and directed towards the lateral lobes of the prostate and or towards the middle lobe. The pressure of the liquid is regulated to such a value permitting cutting and mincing of the softer adenomatous tissue and sparing the harder and more elastic collagen fibers of the prostate capsule. A sweeping motion of the operational tip is effected, such as in painting or brushing the hyperplastic prostate tissue with the liquid jets. In turn cutting and mincing this tissue to a depth of a few millimeters is effected under continuous visual inspection. The converging liquid jets permit cutting the tissue to a predetermined depth of 2 to 3 mm. Bleeding is stopped by contact heating induced by pressing the active face of the heating member against the surface of the bleeding tissue. Heating of the liquid is induced shortly following the activation of the heating unit by which the heating member reaches the temperature of 90 degrees Celsius within a few seconds. Pressing the active face of the heating member against the bleeding tissue for a while stops the bleeding by sealing the walls of injured blood vessels. The ejected liquid surrounding the operational tip and a segment of the tubular body is passively evacuated through the working channel of the cystoscope. The procedure is completed when the prostate capsule is reached. At the end of the procedure the minced prostate tissue is evacuated in a way conventional to the urological practice and sent to histological examination.

1. A surgical instrument utilizing liquid for reducing bleeding from a tissue by contact heating, said surgical instrument comprising
a slender tubular body adapted and arranged for endoscopic procedures;
an operational tip disposed at the distal end of said slender tubular body;
a heating member having an active face associated with said operational tip for conducting heat from a surface of said active face to said tissue, and
wherein said heating member is heatable to a predefined operating temperature.

2. A surgical instrument as in claim 1, wherein said operational tip comprises a lumen which forms a continuum with a lumen of said slender tubular body.

3. A surgical instrument as in claim 2, wherein said operational tip further comprises at least two jetting outlets disposed at a sidewall of said operational tip providing for jetting liquid from said lumen towards said tissue laterally with respect to said operational tip for dissecting through a surface of said tissue.

4. A surgical instrument as in claim 1, further comprising a gripping handle attached to said slender tubular body.

5. A surgical instrument as in claim 4, further comprising means for rotating said jetting outlets relative to said gripping handle.

6. A surgical instrument as in claim 4, further comprising means for independently rotating said active face relative to said gripping handle.

7. A surgical instrument as in claim 3, wherein said at least two jetting outlets converge at a converging point laterally displaced with respect to said operational tip.

8. A surgical instrument as in claim 7, wherein a plane containing the axes of said at least two jetting outlets is oriented relative to the main axis of said operational tip at an angle selected from a range of angles between zero to ninety degrees.

9. A surgical instrument as in claim 7, wherein said operational tip comprises a plurality of converging jetting outlets.

10. A surgical instrument as in claim 1, wherein said heating member comprises any item selected from a group of items consisting of a heating element, a heat buffer and any combination thereof.

11. A method for regulating a temperature of a heating member of a surgical instrument, wherein said surgical instrument comprises an operational tip disposed at the distal end of a slender tubular body, and wherein said heating member associated with said operational tip, said method comprising delivering liquid heated to a predefined temperature through said slender tubular body for heating said heating member.

12. A method for regulating a temperature of a heating member of a surgical instrument as in claim 11, further comprising electrically heating said heating member.

13. A method for regulating a temperature of a heating member as in claim 11, wherein said heating member comprises a heat buffer.

14. A surgical instrument having a gripping handle and utilizing liquid for dissecting and/or mincing and removing at least a portion of a layer of a tissue, said surgical instrument comprising
a slender tubular body for delivering said liquid;
a hollow operational tip disposed at the distal end of said slender tubular body, wherein the lumen of said operational tip forms a continuum with a lumen of said tubular body, said operational tip comprises at least two jetting outlets disposed at a sidewall of said operational tip, and
wherein said jetting outlets provide for jetting said liquid from said lumen towards said tissue laterally with respect to said operational tip for dissecting through a surface of said tissue, and wherein said jetting outlets are rotatable relative to said gripping handle.

15. A surgical instrument as in claim 14, wherein said at least two jetting outlets converge at a converging point laterally displaced with respect to said operational tip.

16. A surgical instrument as in claim 14, wherein a plane containing the axes of said at least two jetting outlets is oriented relative to the main axis of said operational tip at an angle selected from a range of angles between zero to ninety degrees.

17. A surgical instrument as in claim 14, wherein said operational tip comprises a plurality of converging jetting outlets.

18. A surgical instrument as in claim 14, wherein said operational tip further comprises a heating member having an active face for conducting heat from a surface of said active face to said tissue, wherein said heating member is heatable to a predefined operating temperature.

19. A surgical instrument as in claim 18, wherein said heating member comprises any item selected from a group of items consisting of a heating element, a heat buffer and any combination thereof.

20. A surgical instrument as in claim 19, further comprising means for independently rotating said active face relative to said gripping handle.
